(19) United States
(12) Patent Application Publication
(10) Pub. No.: US 2014/0357725 A1
(43) Pub. Date: Dec. 4, 2014

(54) TERPENE ANALOGUES AND USES THEREOF FOR TREATING NEUROLOGICAL CONDITIONS

(71) Applicants: Donald F. Weaver, Halifax (CA); Mark Andrew Reed, Hubley (CA); Alexander McLellan, Ketch Harbour (CA); Shengguo Sun, Beechville (CA)

(72) Inventors: Donald F. Weaver, Halifax (CA); Mark Andrew Reed, Hubley (CA); Alexander McLellan, Ketch Harbour (CA); Shengguo Sun, Beechville (CA)

(73) Assignee: NEUROQUEST INC., Charlottetown, PE (CA)

(21) Appl. No.: 14/232,771

(22) PCT Filed: Jul. 13, 2012
(86) PCT No.: PCT/IB2012/001390

§ 371 Date: Jun. 4, 2014

Related U.S. Application Data
(60) Provisional application No. 61/507,764, filed on Jul. 14, 2011.

(51) Int. Cl. C07C 43/176; C07C 33/38; C07C 43/172; C07C 33/05; C07C 43/162

(52) U.S. Cl. CPC C07C 43/176; C07C 33/05; C07C 43/162; C07C 43/172; C07C 33/38
USPC 514/715; 514/729

(57) ABSTRACT

The present application provides terpene analogues of Formula 1 and methods and uses thereof for treating neurological conditions such as pain in general and neuropathic pain specifically.

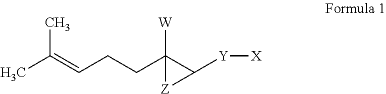

wherein: Y is a C₁ to C₂₀ alkylene, C═O, SO, SO₂, or absent; X is H, OR¹, N—(R²)₂, a C₁ to C₂₀ alkyl, or a heterocyclyl (for example, heteroaryl), wherein when Y is absent X is not H; R¹ is H, a C₁ to C₂₀ alkyl, or a CH₂-aryl; R² is independently H, a C₁ to C₂₀ alkyl, aryl, OR¹, CN or C(═O)—R³; R³ is a substituted or unsubstituted C₁ to C₂₀ alkyl, or a aryl; W is H, C₁ to C₂₀ alkyl, or aryl; and Z is C₁ to C₂₀ alkylene; or a pharmaceutically acceptable isomer, salt or ester thereof. These terpene analogues are useful in treating pain and can also be used to treat other electrical disorders in the central and peripheral nervous system.

### FIELD OF THE INVENTION

The present invention relates to the field of therapies for the treatment of neurological disorders. More specifically, the present invention relates to terpene analogues and uses thereof for treating pain.

### BACKGROUND

Chronic pain, whether nociceptive or neuropathic, is subject to intensive research, with significant resources being devoted to the development of analgesic drugs. Neuropathic pain is notoriously difficult to treat. Current treatments of neuropathic pain include the use of anti-convulsants, anti-depressants, and opioids. They are often either ineffective or result in unacceptable side effects at the doses required for analgesia. A chronic progressive condition that strikes a generally middle aged and older demographic, neuropathic pain rates are expected continue to rise much higher than the current estimate of more than 12 million present day sufferers in North America alone. The chronic pain associated with peripheral neuropathy is known to result in tremendous human suffering, including loss of mobility, lost productivity, difficulty maintaining social and family relationships, and depression. Therefore there is an unmet medical need for the development of novel treatments for neuropathic pain.

Neuropathic pain is produced by damage to, or pathological changes in, the peripheral central nervous system, typically producing pain that is described as “burning”, “electric”, “tingling”, and “shooting” in nature. Other characteristics of neuropathic pain include hyperpathia, hyperesthesia, dysesthesia, and paresthesia.

Voltage-gated sodium channels in sensory neurons play an essential role in several chronic pain neuropathies that arise from injury to peripheral nerves, such as those caused by trauma, nerve compression, diabetic neuropathy, viral infections or chemotherapeutic agents. Compounds that exhibit a use-dependent blockade of these channels, including anti-convulsants, anti-arrhythmics, local anaesthetics, anti-epilepsy drugs, drugs for sleep disorders, anti-migraine drugs and anti depressants, have been found to be effective in the treatment of neuropathic pain and electrical disorders in the central and peripheral nervous system, which in turn provides clinical support for the importance of these channels in such pain states.

Current conventional pharmacological strategies for treating neuropathic pain include sodium channel blockers, tri-cyclic antidepressants, serotonin reuptake inhibitors, anticonvulsants, GABA B receptor inhibitors, NMDA receptor antagonists, and topical agents. TRP (Transient Receptor Potential Vanilloid) antagonists prevent pain by silencing a nociceptor in the periphery where pain is generated. Compounds that act upon the TRP family of receptors can also be used to treat other electrical disorders in the central and peripheral nervous system.

The efficacy of these pharmacological treatments is often limited by side effects at the doses required for analgesia, as well as in some cases long delays before the onset of analgesia, a substantial rate of nonresponsiveness to therapy, and a potential for addiction. Therefore, there is a need for a novel preparation to treat neuropathic pain.

In terms of inhibition of nerve function, a variety of classes of naturally derived compounds has shown the ability to inhibit neuronal firing by various methods, including affects on nerve cell receptors and associated ion channels. For example, flavanoids, terpenes, terpenoids, ginsenosides, and a variety of other dietary and environmental compounds have been shown to influence nerve transmission rates.

Stotz et al. describe a role of citral and the isolated aldehyde and alcohol cis or trans isomers of citral (neral, nerol, geranial, geraniol) as being effective antagonists of TRP ion channels (Stotz et al., Citral Sensing by TRANSient Receptor Potential Channels in Dorsal Root Ganglion Neurons. PLoS ONE (2008), 3(5): e2082).

There remains a need for alternative therapies for treating disorders of nerve cell transmission and, in particular, neuropathic pain.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide terpene analogues and methods and uses thereof for treating neurological conditions, such as pain in general and neuropathic pain specifically. Compounds that are useful in the treatment of pain can also often be used to treat other electrical disorders in the central and peripheral nervous system.

In accordance with an aspect there is provided a method of treating a neurological condition in a subject comprising administering to the subject a terpene analogue of Formula 1:

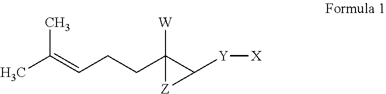

wherein:

Y is a substituted or unsubstituted C₁ to C₂₀ alkylene, C═O, SO, SO₂, or absent;

X is H, OR¹, N—(R²)₂, a substituted or unsubstituted C₁ to C₂₀ alkyl, or a substituted or unsubstituted heterocyclyl (for example, heteroaryl), wherein when Y is absent X is not H;

R¹ is H, a substituted or unsubstituted C₁ to C₂₀ alkyl, or a substituted or unsubstituted CH₂-aryl;

each R² is independently H, a substituted or unsubstituted C₁ to C₂₀ alkyl, aryl, OR¹, CN or C(═O)—R³;

R³ is a substituted or unsubstituted C₁ to C₂₀ alkyl, or a substituted or unsubstituted aryl;

W is H, a substituted or unsubstituted C₁ to C₂₀ alkyl, or a substituted or unsubstituted aryl; and

Z is a substituted or unsubstituted C₁ to C₂₀ alkylene;

or a pharmaceutically acceptable isomer, salt or ester thereof.

In one embodiment, there is provided a terpene analogue of Formula 1a:

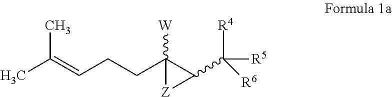

wherein:

R⁴ is OH, alkoxyl, aryloxyl, —C(═O)H, —COOH, —NH₂, —SO₂Aryl, —SO₂NHAryl, —NHSO₂Aryl, —NHalkyl, —N(alkyl)₂, or —NHCO-Aryl;

W, R⁵, and R⁶ are each independently H, alkyl, aryl or alkylaryl, where alkyl is C₁ to C₂₀; and

Z is a C₁ to C₂₀ alkylene.

Isomers can include, for example, syn and anti isomers of the terpene compound.

In accordance with another aspect, there is provided a pharmaceutical composition useful for treating neurological conditions comprising a terpene analogue of Formula 1:

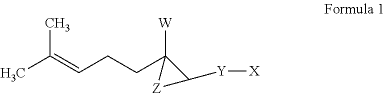

wherein:

Y is a substituted or unsubstituted C₁ to C₂₀ alkylene, C═O, SO, SO₂, or absent;

X is H, OR¹, N—(R²)₂, a substituted or unsubstituted C₁ to C₂₀ alkyl, or a substituted or unsubstituted heterocyclyl (for example, heteroaryl), wherein when Y is absent X is not H;

R¹ is H, a substituted or unsubstituted C₁ to C₂₀ alkyl, or a substituted or unsubstituted CH₂-aryl;

each R² is independently H, a substituted or unsubstituted C₁ to C₂₀ alkyl, aryl, OR¹, CN or C(═O)—R³;

R³ is a substituted or unsubstituted C₁ to C₂₀ alkyl, or a substituted or unsubstituted aryl;

W is H, a substituted or unsubstituted C₁ to C₂₀ alkyl, or a substituted or unsubstituted aryl; and

Z is a substituted or unsubstituted C₁ to C₂₀ alkylene;

or a pharmaceutically acceptable isomer, salt or ester thereof

In accordance with one embodiment the pharmaceutical composition useful for treating neurological conditions comprises a terpene analogue of Formula 1a:

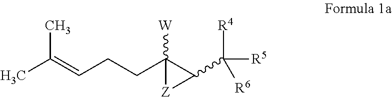

wherein:

R⁴ is OH, alkoxyl, aryloxyl, —C(═O)H, —COOH, —NH₂, —SO₂Aryl, —SO₂NHAryl, —NHSO₂Aryl, —NHalkyl, —N(alkyl)₂, or —NHCO-Aryl;

W, R⁵, and R⁶ are each independently H, alkyl, aryl or alkylaryl, where alkyl is C₁ to C₂₀; and

Z is a C₁ to C₂₀ alkylene.

### BRIEF DESCRIPTION OF THE FIGURES

FIG. 1 shows a sodium channel patch clamp assay having a representative inhibition curve for compound OBM 2979.

FIG. 2 shows a plot of percentage sodium current versus concentration of OBM 2979 vs control.

FIG. 3 illustrates Ca²⁺ imaging of OBM 2983 at various concentrations in the presence of HEK-TRPV cells.

FIG. 4 shows a dose response curve of Zebra Fish embryo assay for OBM 2979.

### DETAILED DESCRIPTION

Unless defined otherwise, all technical and scientific terms used herein have the same meaning as commonly understood by one of ordinary skill in the art to which this invention belongs.

Unless the context clearly indicates otherwise, as used herein plural forms of the terms herein are to be construed as including the singular form and vice versa.

The terms “comprises” and “comprising” as used herein will be understood to mean that the list following is non-exhaustive and may or may not include any other additional suitable items, for example one or more further feature(s), component(s) and/or ingredient(s) as appropriate.

It should be noted that if the stereochemistry of a structure or a portion of a structure is not indicated with, for example, bold or dashed lines, the structure or the portion of the structure is to be interpreted as encompassing all stereoisomers of it. Curved or “squiggled” bond lines in structures or portions thereof are to be interpreted to encompass all cis and trans stereoisomers. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit.

As used herein, “neuropathic pain” refers to pain caused by various types of nerve damage. Some examples of neuropathic pain conditions that can be treated by the method of the present invention include, but are not limited to, diabetic peripheral neuropathy, herpes zoster, post herpetic neuralgia, trigeminal neuralgia, complex regional pain syndrome, reflex sympathetic dystrophy, migraine headache, phantom limb syndrome, neuropathic pain due to chronic disease (multiple sclerosis, HIV, etc), neuropathic pain due to trauma (causalgia), neuropathic pain due to impingement (i.e. sciatica, carpal tunnel, etc.), neuropathic pain due to drug exposure or toxic chemical exposure, neuropathic pain due to infection or post infection, neuropathic pain due to impaired organ function, neuropathic pain due to vascular disease, neuropathic pain due to metabolic disease, neuropathic pain due to cancer or cancer treatment, neuropathic pain due to autoimmune disease, neuropathic pain due to fibromylagia, and neuropathic pain with no known cause (idiopathic).

As used herein, a “terpene compound” refers to a terpene, a terpenoid, or a pharmaceutically acceptable isomer, salt, ester or solvate thereof. Isomers can include, for example, (Z)- or (E)-isomers of the terpene compound. As used herein, a “terpenoid” refers to a chemically modified terpene. Examples of terpenoids include, but are not limited to, terpenoid aldehydes, terpenoid acids, terpenoid esters and terpenoid oxides.

As used herein, a “terpene analogue” is a compound that is an analogue of a terpene compound or a terpenoid.

As used herein, “aliphatic” refers to hydrocarbon moieties that are linear, branched or cyclic, may be alkyl, alkenyl or alkynyl, may be substituted or unsubstituted and may include one or more heteroatoms. “Alkyl” means a monovalent straight, branched, or cyclic hydrocarbon radical, e.g., C_{f}H_{2f+1}, where f is an integer, which may include one or more heteroatoms. For example, an alkyl is a C₁-C₂₀ monovalent straight, branched, or cyclic hydrocarbon radical. The term “alkyl” encompasses cycloalkyl, heteroalkyl and heterocyclyl moieties. “Alkenyl” means a hydrocarbon moiety that is linear, branched or cyclic and comprises at least one carbon to carbon double bond, which may include one or more heteroatoms. “Alkynyl” means a hydrocarbon moiety that is linear, branched or cyclic and comprises at least one carbon to carbon triple bond, which may include one or more heteroatoms.

“Aryl” means a moiety including a substituted or unsubstituted aromatic ring, including heteroaryl moieties and moieties with more than one conjugated aromatic ring; optionally it may also include one or more non-aromatic ring. “C₅ to C₈ Aryl” means a moiety including a substituted or unsubstituted aromatic ring having from 5 to 8 carbon atoms in one or more conjugated aromatic rings. Examples of aryl moieties include phenyl.

“Alkylene” means a substituted or unsubstituted divalent alkyl radical, e.g., —C_{f}H_{2f}— wherein f is an integer. “Alkenylene” means a divalent alkenyl radical, e.g., —CHCH—. An alkylene may include one or more heteroatoms. For example, an “alkylene” is a C₁-C₂₀ divalent straight, branched, or cyclic hydrocarbon.

“Heterocyclyl” means a moiety including a substituted or unsubstituted cyclic radical having from 2 to 8 carbon atoms and at least one heteroatom in one or more rings. As used herein, “heteroatom” refers to non-carbon and non-hydrogen atoms, such as, for example, O, S, and N. Examples of non-aromatic heterocyclic moieties include imidazolidinyl, pyrazolidinyl, oxazolidinyl and dioxanyl. Included in the term “heterocyclyl” are “heteroaryl” moieties. “Heteroaryl” means a moiety including a substituted or unsubstituted aromatic ring having from 3 to 8 carbon atoms and at least one heteroatom in one or more conjugated aromatic rings. Examples of heteroaryl moieties include pyridyl, furanyl, thienyl, imidazolyl, pyrazolyl, thiazolyl, isothiazolyl, oxadiazolyl.

“Substituted” means having one or more substituent moieties whose presence does not interfere with the desired function or reactivity. Examples of substituents include alkyl, alkenyl, alkynyl, cycloalkyl, aryl, heteroaryl, hydroxyl, alkoxyl, amino, alkylamino, alkenylamino, amide, thioether, alkylcarbonyl, alkylcarbonyloxy, alkoxycarbonyloxy, carbonate, alkoxycarbonyl, aminocarbonyl, alkylthiocarbonyl, halo (such as fluoro, chloro or bromo), acylamino, imino, sulfhydryl, alkylthio, thiocarboxylate, dithiocarboxylate, sulfate, sulfato, sulfonate, sulfamoyl, sulfonamide, nitro, nitrile, azido, heterocyclyl, ether, ester, thioester, or a combination thereof. The substituents may themselves be substituted. For instance, an amino substituent may itself be mono or independently disubstituted by further substituents defined above, such as alkyl, alkenyl, alkynyl, and cycloalkyl.

As used herein, the term “composition” can refer to a pharmaceutical preparation containing a terpene compound alone or in combination with a pharmaceutically acceptable diluent or excipient. The pharmaceutical composition of the present invention can be prepared using standard, well known techniques. Pharmaceutical compositions of the present invention do not necessarily require inclusion of any pharmaceutically acceptable diluent or excipient. However, such diluents or excipients can be incorporated into the composition as required depending on the desired characteristics of the composition.

As described above, various terpenes and terpenoids are known to have therapeutic properties. The present application relates to terpene analogues that have also been found to be therapeutically useful, for example, in the treatment of pain.

TRP (Transient Receptor Potential Vanilloid) antagonists prevent pain by silencing a nociceptor in the periphery where pain is generated. Without wishing to be bound by theory or mechanism, the terpene analogues described herein have been found to be useful for treating disorders of nerve transmission, such as neuropathic pain, by restoring the balance between nerve excitation and inhibition. This may be achieved by affecting the activity of neuronal channels, such as sodium ion channels and TRP.

In accordance with one aspect there is provided a method of treating a neurological condition in a subject comprising administering to the subject a terpene analogue of Formula 1:

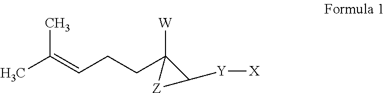

wherein:

Y is a substituted or unsubstituted C₁ to C₂₀ alkylene, C═O, SO, SO₂, or absent;

X is H, OR¹, N—(R²)₂, a substituted or unsubstituted C₁ to C₂₀ alkyl, or a substituted or unsubstituted heterocyclyl (for example, heteroaryl), wherein when Y is absent X is not H;

R¹ is H, a substituted or unsubstituted C₁ to C₂₀ alkyl, or a substituted or unsubstituted CH₂-aryl;

each R² is independently H, a substituted or unsubstituted C₁ to C₂₀ alkyl, aryl, OR¹, CN or C(═O)—R³;

R³ is a substituted or unsubstituted C₁ to C₂₀ alkyl, or a substituted or unsubstituted aryl;

W is H, a substituted or unsubstituted C₁ to C₂₀ alkyl, or a substituted or unsubstituted aryl; and

Z is a substituted or unsubstituted C₁ to C₂₀ alkylene;

or a pharmaceutically acceptable isomer, salt or ester thereof.

In one embodiment, there is provided a terpene analogue of Formula 1a:

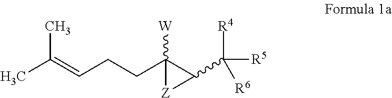

wherein:

R⁴ is OH, alkoxyl, aryloxyl, —NH₂, —SO₂Aryl, —SO₂NHAryl, —NHSO₂Aryl, —NHalkyl, —N(alkyl)₂, or —NHCO-Aryl;

W, R⁵, and R⁶ are each independently H, a substituted or unsubstituted C₁ to C₂₀ alkyl, a substituted or unsubstituted aryl or a substituted or unsubstituted alkylaryl; and

Z is a substituted or unsubstituted C₁ to C₂₀ alkylene;

or a pharmaceutically acceptable isomer, salt or ester thereof.

In another alternative embodiment, there is provided a terpene analogue of Formula 1 wherein:

Y is a absent;

X is —C(═O)H, —COOH, —SO₂Aryl, or —SO₂NHAryl,

W is H, a substituted or unsubstituted C₁ to C₂₀ alkyl, a substituted or unsubstituted aryl or a substituted or unsubstituted alkylaryl; and

Z is a substituted or unsubstituted C₁ to C₂₀ alkylene;

or a pharmaceutically acceptable isomer, salt or ester thereof.

In an alternative embodiment, there is provided a terpene analogue of Formula 1 wherein:

Y is a C₁ to C₆ alkylene, C═O, SO, or SO₂;

X is H, OR¹, N—(R²)₂, a substituted or unsubstituted C₁ to C₆ alkyl, or a substituted or unsubstituted 4 to 6 membered heterocyclyl (for example, heteroaryl);

R¹ is H, a substituted or unsubstituted C₁ to C₆ alkyl, or a substituted or unsubstituted CH₂-aryl;

R² is independently H, a substituted or unsubstituted C₁ to C₆ alkyl, aryl, OR¹, CN or C(═O)—R³;

R³ is a substituted or unsubstituted C₁ to C₆ alkyl, or a substituted or unsubstituted aryl;

W is H, C₁ to C₆ alkyl, or aryl; and

Z is C₁ to C₆ alkylene;

or a pharmaceutically acceptable isomer, salt or ester thereof

In another alternative embodiment, there is provided a terpene analogue of Formula 1 wherein:

Y is a absent;

X is a substituted or unsubstituted 4 to 6 membered non-aromatic heterocyclyl or a substituted or unsubstituted 4 to 6 membered aromatic heterocycle (for example, heteroaryl);

W is H or a substituted or unsubstituted C₁ to C₆ alkyl; and

Z is a substituted or unsubstituted C₁ to C₆ alkylene;

or a pharmaceutically acceptable isomer, salt or ester thereof

In accordance with certain embodiments W is methyl or phenyl and Z is methylene.

Exemplary terpene analogue in accordance with the present invention include monterpenoid analogs of 3,7-dimethylocta-2,6-dien-1-ol. These are shown in Table 1.

TABLE 1

| ID | | | |
|---|---|---|---|
| Number | Terpene analogue structure | Properties | Name |
| 2979 | 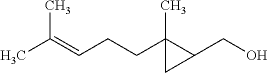 | Chemical Formula: C₁₁H₂₀O Exact Mass: 168.15 | (2-methyl-2-(4- methylpent-3-en-1- yl)cyclopropyl)methanol |
| 2989 | 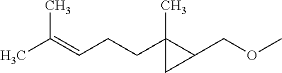 | Chemical Formula: C₁₂H₂₂O Exact Mass: 182.17 | 2-(methoxymethyl)-1- methyl-1-(4-methylpent-3- en-1-yl)cyclopropane |
| 2993 | 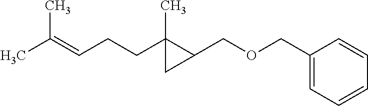 [SMILES: CC(C)=CCCC1(C)CC1COCc1ccccc1] | Chemical Formula: C₁₈H₂₆O Exact Mass: 258.20 | (((2-methyl-2-(4- methylpent-3-en-1- yl)cyclopropypmethoxy) methyl)benzene |
| 2994 | 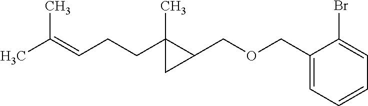 [SMILES: CC(C)=CCCC1(C)CC1COCc1ccccc1Br] | Chemical Formula: C₁₈H₂₅BrO Exact Mass: 336.11 | 1-bromo-2-(((2-methyl-2- (4-methylpent-3-en-1- yl)cyclopropyl)methoxy) methyl)benzene |
| 2995 | 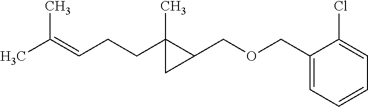 [SMILES: CC(C)=CCCC1(C)CC1COCc1ccccc1Cl] | Chemical Formula: C₁₈H₂₅ClO Exact Mass: 292.16 | 1-chloro-2-(((2-methyl-2- (4-methylpent-3-en-1- yl)cyclopropyl)methoxy) methyl)benzene |
| 2999 | 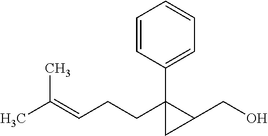 [SMILES: CC(C)=CCCC1(c2ccccc2)CC1CO] | Chemical Formula: C₁₆H₂₂O Exact Mass: 230.17 | (2-(4-methylpent-3-en-1- yl)-2-phenylcyclopropyl) methanol |

The terpene compounds of Formula 1 and 1a, or corresponding pharmaceutically acceptable salts, esters or solvates thereof, can be used as active components in compositions for administration to a subject for treating a neurological condition. The term “solvate” is intended to include “hydrate”. These compositions are not natural oils derived as distillates of plant material, however, the terpene compounds of Formula 1 and 1a used to prepare the synthetic compositions can include one or more compounds that have been isolated from plant material.

The pharmaceutical composition comprises a terpene analogue of Formula 1 or 1a, as described above, in amount effective to influence the balance between nerve excitation and inhibition. It has been found that affecting the activity of both sodium gated ion channels and/or TRP channels can be useful for treating disorders of nerve transmission, such as neuropathic pain, by restoring the balance between nerve excitation and inhibition.

The therapeutic terpene compounds can be formulated for administration to a subject by a route that is effective for delivering the compound and, thereby, restoring the balance between nerve excitation and inhibition by affecting the activity of both sodium ion channels and TRP channels. Suitable routes of administration include intravenous, topical, oral, intranasal, intravaginal and intrarectal. The therapeutic compounds may be administered with a pharmaceutically acceptable vehicle.

The compositions described herein can be prepared and administered in a wide variety of dosage forms, such as, but not limited to, a suspension, pill, gel, oil, cream, patch, spray or aerosol. The composition can be formulated to be suitable for oral administration, topical administration, intranasal, transdermal, intravaginal, and intrarectal administration. Processes for manufacture of such compositions are briefly described below, however, the techniques employed in these processes are standard and well known to a worker skilled in the art. It will be obvious to those skilled in the art that the following dosage forms can comprise as the active component, a terpene compound of Formula 1 or 1a, a corresponding pharmaceutically acceptable salt, ester or solvate thereof, or any combination thereof. In certain embodiments, the composition comprises a combination of two or more terpene compounds of Formula 1 or 1a.

For preparing pharmaceutical compositions from the terpene compounds, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component.

In tablets, the active component is mixed with the carrier having the necessary binding properties in suitable proportions and compacted in the shape and size desired. Some examples of suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. Liquid preparations for parenteral injection can be formulated in solution in aqueous polyethylene glycol solution.

Aqueous solutions suitable for oral use can be prepared by dissolving the active component in water and adding suitable colorants, flavors, stabilizing and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

A particularly preferred mode of administration of the composition comprising a terpene compound as described herein, is to a skin surface via a topical route. Such a composition is topically applied in the form of a lotion, solution, cream, ointment or powder. For example, the composition can be formulated into a cream consisting of an aqueous emulsion of polyethylene glycols or liquid paraffin or can be incorporated at a concentration between 1 and 10% into an ointment consisting of a white wax or white soft paraffin base together with such stabilizers and preservatives as may be required. The topical compositions can contain additional ingredients such as binders, excipients, antioxidants, and dyes.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted creams, lotions, ointments, tablets, capsules, or powders in tubes, vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The dosages, however, may be varied depending upon the requirements of the patient, the severity of the condition being treated, and the compound being employed. Determination of the proper dosage for a particular situation is within the skill of the art. Generally, treatment is initiated with smaller dosages which are less than the optimum dose of the compound. Thereafter, the dosage is increased by small increments until the optimum effect under the circumstances is reached. For convenience, the total daily dosage may be divided and administered in portions during the day, if desired.

Terpene compounds as described herein are useful for treating disorders of nerve transmission by restoring the balance between nerve excitation. This can be achieved by affecting the activity of neuronal channels, such as sodium ion channels and TRP channels.

The activity of the terpene compounds, including their ability to affect nerve transmission, can be evaluated using different assays known in the art. For example, assays which may be particularly useful include the sodium channel patch clamp, the zebrafish anaesthesia assay, and/or a TRPV 1 assay.
a) Sodium Channel—Changes in neuronal excitability as a result of alteration of ion channel activity and/or function by a bioactive substance can be examined using typical slices taken from the rodent brain or spinal cord.
b) Zebrafish Anaesthesia Assay—The zebrafish (Danio rerio) model organism is increasingly used for assessing drug toxicity and safety. Numerous studies now confirm that mammalian and zebrafish toxicity profiles are strikingly similar. We have found, using a tailored Zebrafish assay, that this assay is a vertebrate model which can be utilized as a screening tool for analgesic activity.
c) TRPV1 Assay—TRPV1 (Transient Receptor Potential Vanilloid, Type 1) is a member of the transient receptor potential (TRP) family of ion channels. These channels mediate numerous sensory interactions, including nociception, inflammation, and their modulation is useful in a number of related pathologies, pain being one example. Thus, modulation of TRPV1 is therefore an attractive prospect for drug development in the field of analgesia. Because TRP channels are selective for calcium ions, the uptake of Ca²⁺ provides a basis for the development of a functional assay to assess ligand potency.

Various terpene compounds as described herein have been tested by bath application of known receptor antagonists and agonists to examine for changes in excitability and/or attenuation of ion channels, for the purpose of elucidating a mechanism of action. The terpene compounds show significant ability to reduce membrane currents and early indication associated with the analgesic effects. In addition, patch clamp testing has shown that the compounds have a strong effect on sodium channel currents measured in dorsal root ganglion neurons. Voltage gated sodium channels are known to be relevant drug targets for neuropathic pain, as this family of ion channels governs the generation of action potential firing. (Josephine Lai, John C Hunter, Frank Porreca, The role of voltage-gated sodium channels in neuropathic pain Current Opinion in Neurobiology, Volume 13, Issue 3, June 2003, Pages 291-297).

Zebrafish embryos were tested, at various concentrations, to establish and identify conditions and phenotypic readouts (e.g. touch response, swim behavior) that could be used as an indicator of analgesic actively. The terpene compounds described herein were found to inhibit touch response in a dose dependent and reversible manner. Further, compounds in accordance with the present invention show various degrees of agonist and antagonist activity at the TRPV 1 channel.

To gain a better understanding of the invention described herein, the following examples are set forth. It should be understood that these examples are for illustrative purposes only. Therefore, they should not limit the scope of this invention in any way.

### EXAMPLES

### Example 1

### Synthesis of 2-methyl-2-(4-methylpent-3-en-1-yl)cyclopropyl)methanol

2-Methyl-2-(4-methylpent-3-en-1-yl)cyclopropyl)methanol, shown below and identified here in as “OBM 2979” was synthesised according to the process shown in Scheme 1.

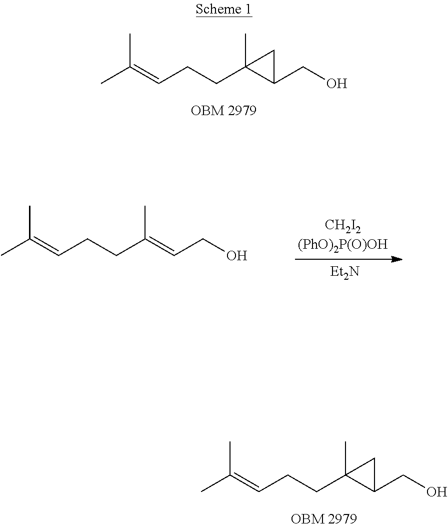

To a solution of 2.92 g (11.7 mmol) diphenylphosphate in 30 ml dry dichloromethane was added 1.2 ml (11.7 mmol) diethylzinc and 0.94 ml (11.7 mmol) diiodomethane with cooling under −10° C. under argon atmosphere. The solution was stirred for 2 hours before adding 1.2 ml (9.7 mmol) geraniol. The reaction was stirred overnight, recovering to room temperature. The run was quenched with 10 ml 1M phosphorus acid and washed with water (10 ml). The mixture was dried over anhydrous sodium sulfate. 2-Methyl-2-(4-methylpent-3-enyl)cyclopropyl)methanol (1.3 g, 80%) was obtained by flash column chromatography (20% ethyl acetate in hexanes).

¹H NMR (500 MHz, CDCl₃) δ (ppm) 0.13 (t, J=4.9 Hz, 1H), 0.50 (dd, J=8.6, 4.5 Hz, 1H), 0.90 (m, 1H), 1.21 (s, 3H), 1.34 (m, 1H), 1.37 (m, 1H), 1.40 (m, 1H), 1.61 (s, 3H), 1.62 (s, 3H), 2.05 (m, 2H), 3.45 (m, 1H), 3.70 (m, 1H), 5.10 (t, J=7.1 Hz, 1H).

¹³C NMR (125 MHz, CDCl₃): δ (ppm) 17.5, 18.1, 18.2, 20.3, 25.9, 26.2, 26.7, 41.5, 64.4, 125.1, 131.8.

### Example 2

### Synthesis of Alkoxy Compounds

The following compounds were synthesised according to Scheme 2

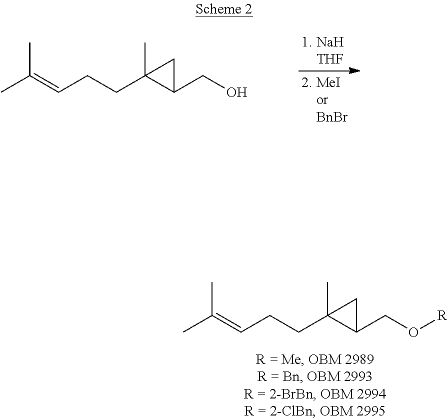

### 2-(methoxymethyl)-1-methyl-1-(4-methylpent-3-enyl)cyclopropane

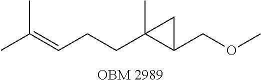

To a suspension of 0.26 g (10.7 mmol) sodium hydride in 15 ml dry NMP was added 0.60 g (3.6 mmol) (2-methyl-2-(4-methylpent-3-enyl)cyclopropyl)methanol under argon atmosphere. After 10 min, 0.29 ml (4.64 mmol) iodomethane was added. The reaction was stirred overnight before quenching with 20 ml water. The mixture was extracted with ethyl acetate (2×20 ml) and dried over anhydrous sodium sulfate. 2-(methoxymethyl)-1-methyl-1-(4-methylpent-3-enyl)cyclopropane (0.40 g, 62%) was obtained by flash column chromatography (2% ethyl acetate in hexanes).

¹H NMR (500 MHz, CDCl₃) δ (ppm) 0.14 (t, J=4.9 Hz, 1H), 0.55 (dd, J=8.7, 4.4 Hz, 1H), 0.88 (m, 1H), 1.10 (s, 3H), 1.29 (m, 2H), 1.65 (s, 3H), 1.70 (s, 3H), 2.08 (dd, J=8.0, 15.5 Hz, 2H), 3.37 (m, 4H), 3.46 (dd, J=10.4, 6.7 Hz, 1H), 5.13 (m, 1H).

¹³C NMR (125 MHz, CDCl₃): δ (ppm) 17.7, 18.1, 18.2, 20.3, 23.5, 25.8, 26.2, 41.8, 58.7, 73.9, 125.1, 131.6.

### ((2-methyl-2-(4-methylpent-3-enyl)cyclopropyl)methoxy)methyl)benzene

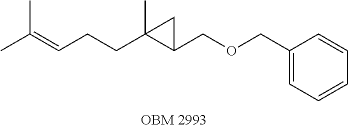 [SMILES: CC(C)=CCCC1(C)CC1COCc1ccccc1]

To a suspension of 0.20 g (9.0 mmol) sodium hydride in 15 ml dry NMP was added 0.60 g (3.6 mmol) (2-methyl-2-(4-methylpent-3-enyl)cyclopropyl)methanol under argon atmosphere. After 10 min, 0.40 ml (3.6 mmol) benzyl bromide was added. The reaction was stirred overnight before quenching with 20 ml water. The mixture was extracted with ethyl acetate (2×20 ml) and dried over anhydrous sodium sulfate. (((2-methyl-2-(4-methylpent-3-enyl)cyclopropyl) methoxy)methyl)benzene (0.60 g, 65%) was obtained by flash column chromatography (3% ethyl acetate in hexanes).

¹H NMR (500 MHz, CDCl₃) δ (ppm) 0.15 (t, J=4.9 Hz, 1H), 0.57 (dd, J=8.7, 4.5 Hz, 1H), 0.95 (m, 1H), 1.09 (s, 3H), 1.28 (m, 2H), 1.65 (s, 3H), 1.70 (s, 3H), 2.08 (dd, J=7.7, 13.7 Hz, 2H), 3.45 (dd, J=7.9, 10.4 Hz, 1H), 3.57 (dd, J=10.4, 6.6 Hz, 1H), 4.56 (d, J=12.1 Hz, 1H), 4.60 (d, J=12.1 Hz, 1H), 5.14 (m, 1H). 7.32 (m, 1H), 7.40 (m, 4H).

¹³C NMR (125 MHz, CDCl₃): δ (ppm) 17.8, 18.1, 18.3, 20.3, 23.8, 25.8, 26.2, 41.8, 71.5, 72.9, 125.1, 127.9, 128.1, 128.3, 128.8, 128.9, 132.6, 139.2.

### 1-bromo-2-(((2-methyl-2-(4-methylpent-3-enyl)cyclopropyl)methoxy)methyl)benzene (2)

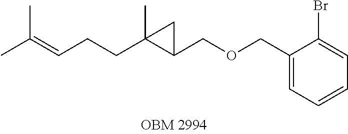 [SMILES: CC(C)=CCCC1(C)CC1COCc1ccccc1Br]

To a suspension of 0.17 g (7.1 mmol) sodium hydride in 15 ml dry NMP was added 0.40 g (2.4 mmol) (2-methyl-2-(4-methylpent-3-enyl)cyclopropyl)methanol under argon atmosphere. After 10 min, 0.71 g (2.8 mmol) 1-(bromomethyl)-2-bromobenzene was added. The reaction was stirred overnight before quenching with 20 ml water. The mixture was extracted with ethyl acetate (2×20 ml) and dried over anhydrous sodium sulfate. 1-Bromo-2-(((2-methyl-2-(4-methylpent-3-enyl)cyclopropyl)methoxy)methyl)benzene (0.60 g, 75%) was obtained by flash column chromatography (2% ethyl acetate in hexanes).

¹H NMR (500 MHz, CDCl₃) δ (ppm) 0.17 (t, J=4.9 Hz, 1H), 0.59 (dd, J=8.7, 4.5 Hz, 1H), 1.01 (m, 1H), 1.12 (s, 3H), 1.30 (m, 2H), 1.64 (s, 3H), 1.70 (s, 3H), 2.11 (dd, J=8.0, 14.5 Hz, 2H), 3.51 (dd, J=8.1, 10.5 Hz, 1H), 3.67 (dd, J=10.5, 6.4 Hz, 1H), 4.62 (d, J=5.6 Hz, 2H), 5.15 (m, 1H), 7.18 (m, 1H), 7.35 (m, 1H), 7.56 (m, 2H).

¹³C NMR (125 MHz, CDCl₃): δ (ppm) 17.8, 18.3, 20.4, 23.6, 25.8, 41.7, 72.1, 123.0, 125.1, 127.8, 127.8, 129.1, 129.3, 129.4, 129.5, 132.9, 133.0, 138.6.

### 1-chloro-2-(((2-methyl-2-(4-methylpent-3-enyl)cyclopropyl)methoxy)methyl)benzene

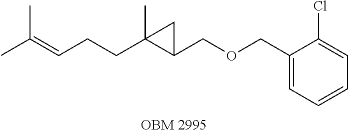 [SMILES: CC(C)=CCCC1(C)CC1COCc1ccccc1Cl]

To a suspension of 0.28 g (11.9 mmol) sodium hydride in 15 ml dry NMP added 0.50 g (3.0 mmol) (2-methyl-2-(4-methylpent-3-enyl)cyclopropyl)methanol under argon atmosphere. After 10 min, 0.73 g (3.6 mmol) 1-(bromomethyl)-2-chlorobenzene was added. The reaction was stirred overnight before quenching with 20 ml water. The mixture was extracted with ethyl acetate (2×20 ml) and dried over anhydrous sodium sulfate. 1-Chloro-2-(((2-methyl-2-(4-methylpent-3-enyl)cyclopropyl)methoxy)methyl)benzene OBM 2995 (0.50 g, 57%) was obtained by flash column chromatography (2% ethyl acetate in hexanes).

¹H NMR (500 MHz, CDCl₃) δ (ppm) 0.14 (t, J=4.8 Hz, 1H), 0.55 (dd, J=8.7, 4.5 Hz, 1H), 0.96 (m, 1H), 1.08 (s, 3H), 1.28 (m, 2H), 1.60 (s, 3H), 1.67 (s, 3H), 2.07 (dd, J=7.7, 15.5 Hz, 2H), 3.46 (dd, J=10.3, 8.2 Hz, 1H), 3.62 (dd, J=10.3, 6.5 Hz, 1H), 4.62 (m, 2H), 5.15 (t, J=7.0 Hz, 1H), 7.20-7.34 (m, 3H), 7.52 (dd, J=7.3 Hz, 1H).

¹³C NMR (125 MHz, CDCl₃): δ (ppm) 17.8, 18.3, 20.4, 23.6, 25.8, 41.7, 72.1, 123.0, 125.1, 127.8, 127.2, 128.8, 129.3, 129.6, 131.6, 133.2, 137.0.

### Example 3

### Synthesis of (2-(4-methylpent-3-enyl)-2-phenylcyclopropyl)methanol

(2-(4-Methylpent-3-enyl)-2-phenylcyclopropyl)methanol was synthesised according to the process shown in Scheme 3.

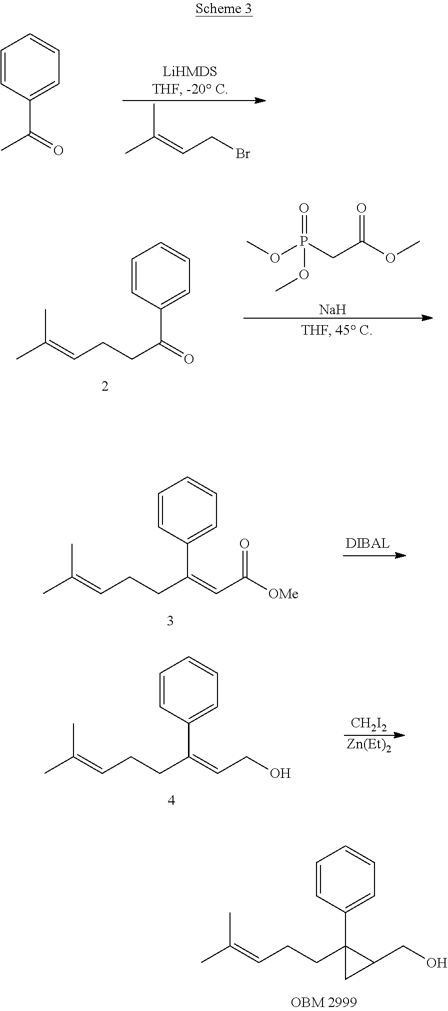

### (z)-7-methyl-3-phenylocta-2,6-dien-1-ol

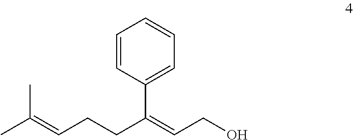 [SMILES: CC(C)=CCC/C(=C/CO)c1ccccc1]

To a solution of 0.5 g (1.9 mmol) (Z)-methyl 7-methyl-3-phenylocta-2,6-dienoate in 15 ml dry toluene was added 5.8 ml (5.8 mmol) diisobutylaluminium hydride at −78° C. under argon atmosphere. The reaction was stirred for 1 hour before quenching with 5 ml methanol. The mixture was added in 50 ml 2 N hydrochloric acid solution and extracted with ethyl acetate (2×20 ml), and dried over anhydrous sodium sulfate. (Z)-7-methyl-3-phenylocta-2,6-dien-1-ol (0.32 g, 78%) was obtained by flash column chromatography (25% ethyl acetate in hexanes).

¹H NMR (500 MHz, CDCl₃) δ (ppm) 1.29 (s, 1H), 1.56 (s, 3H), 1.64 (s, 3H), 2.06 (dd, J=7.3, 15.1 Hz, 2H), 2.98 (t, J=7.4 Hz, 2H), 4.08 (d, J=6.8 Hz, 2H), 5.13 (m, 1H), 5.73 (t, J=6.8 Hz, 1H), 7.16 (d, J=6.9 Hz, 2H), 7.29 (m, 1H), 7.37 (t, J=7.6 Hz, 2H).

¹³C NMR (125 MHz, CDCl₃): δ (ppm) 18.2, 26.1, 27.1, 39.4, 60.7, 124.1, 126.0, 127.5, 128.5, 128.6, 132.4, 140.4, 145.0.

### (2-(4-methylpent-3-enyl)-2-phenylcyclopropyl)methanol

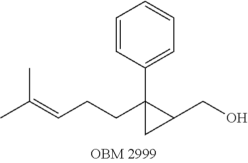 [SMILES: CC(C)=CCCC1(c2ccccc2)CC1CO]

To a solution of 0.42 g (1.6 mmol) diphenylphosphate in 15 ml dry dichloromethane added 1.6 ml (1.6 mmol) diethylzinc, and 0.14 ml (11.7 mmol) diiodomethane cooling under −10° C. under argon atmosphere after 20 min. The solution was stirred for 1 hour before adding 0.3 g (1.3 mmol) (z)-7-methyl-3-phenylocta-2,6-dien-1-ol. The reaction was stirred overnight recovering at room temperature. The run was quenched with 5 ml 1N hydrochloric acid and washed with water (10 ml). The mixture was dried over anhydrous sodium sulfate. (2-(4-methylpent-3-enyl)-2-phenylcyclopropyl)methanol (OBM 2999) (0.27 g, 84%) was obtained by flash column chromatography (20% ethyl acetate and 5% ethyl ether in hexanes).

¹H NMR (500 MHz, CDCl₃) δ (ppm) 0.90 (ms, 2H), 1.21 (m, 1H), 1.26 (s, 1H), 1.34 (m, 1H), 1.51 (s, 3H), 1.66 (s, 3H), 1.89 (m, 2H), 2.04 (m, 1H), 3.29 (m, 2H), 5.04 (m, 1H), 7.26 (m, 1H), 7.33 (m, 4H).

### Example 4

### Sodium (Na⁺) Channel Analysis in Rat DRG Neurons Using Whole Cell Patch-Clamp Techniques

Isolated DRG neurons were suspended in primary neuron basal media and placed on glass coverslips for incubation in humidified atmosphere of 5% CO₂ at 37° C. Coverslip carrying cells was transferred to the bath of an inverted microscope (Zeiss), continuously perfused with oxygenated artificial cerebro-spinal fluid (ACSF) containing (in mM) 124 NaCl, 2.5 KCl, 2 CaCl₂, 1 MgSO₄, 25 NaHCO₃, 1 NaH₂PO₄, and 10 glucose, at a rate of 2-3 ml/min. Recording of whole-cell membrane currents were made at room temperature. Recording pipette (4-6 MO) was filled with internal solution containing (in mM) 145 K-gluconate, 5 NaCl, 1 MgCl₂, 0.2 EGTA, 10 HEPES, 2 Mg-ATP, 0.1 Na-GTP, and 10 phosphocreatine. To isolate Na⁺ currents, DRG neurons were superfused with ACSF containing tetraethylammonium chloride (TEA) 5 mM, cesium chloride (CsCl) 100 μM and cadmium chloride (CdCl) 1 mM, to block potassium and calcium currents. OBM compounds were freshly dissolved in ASCF containing TEA, CsCl and CdCl, prior application via the bath.

For recording Na⁺ currents, cells were held at −60 mV before applying a conditioning hyperpolarizing step (50 ms) to −90 mv to reactivate the voltage-gated Na⁺ channels. The conditioning pulse was followed by depolarizing (150 ms) test pulses to 50 mV in 10 mV increments. Na⁺ currents were recorded in absence, after 3 min in presence of the drugs and after a recovery time of 3 min.

IC₅₀ values were measured and the observed ranges are shown in Table 2.

TABLE 2
Measured IC₅₀s

| | | IC₅₀ |
|---|---|---|
| ID | Structure | (mM) |
| 2979 | 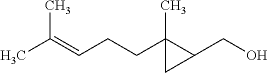 | A |
| 2989 | 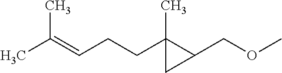 | C |
| 2993 | 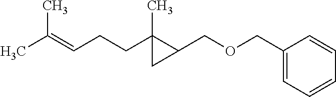 [SMILES: CC(C)=CCCC1(C)CC1COCc1ccccc1] | D |
| 2994 | 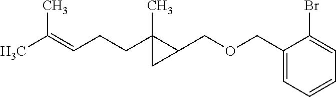 [SMILES: CC(C)=CCCC1(C)CC1COCc1ccccc1Br] | D |
| 2995 | 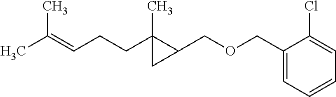 [SMILES: CC(C)=CCCC1(C)CC1COCc1ccccc1Cl] | D |
| 2999 | 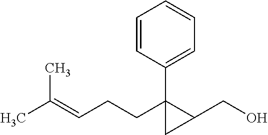 [SMILES: CC(C)=CCCC1(c2ccccc2)CC1CO] | B |

IC₅₀ ranges
A = 0.1-1 mM
B = 1-5 mM
C = 5-10 mM
D = <10 mM

FIG. 1 shows a sodium channel patch clamp assay. The figure shows a representative inhibition curve for compound OBM 2979 and a plot of percentage sodium current versus concentration of OBM 2979 vs control. Calculated IC₅₀-0.7 mM

### Example 5

### Zebrafish Response Assay

Recent results indicate that certain zebrafish embryonic phenotypic readouts, reduced touch response and reduced spontaneous coiling, correlate with analgesic activity, providing an invaluable in vivo vertebrate preclinical bioassay for the identification and characterization of the activity of compounds capable of regulating neuropathic pain (data not shown).

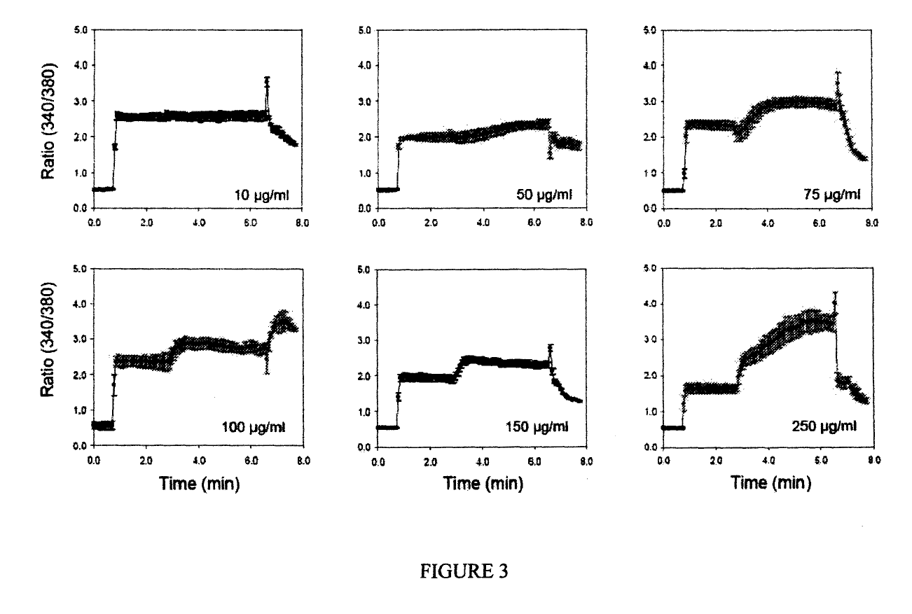

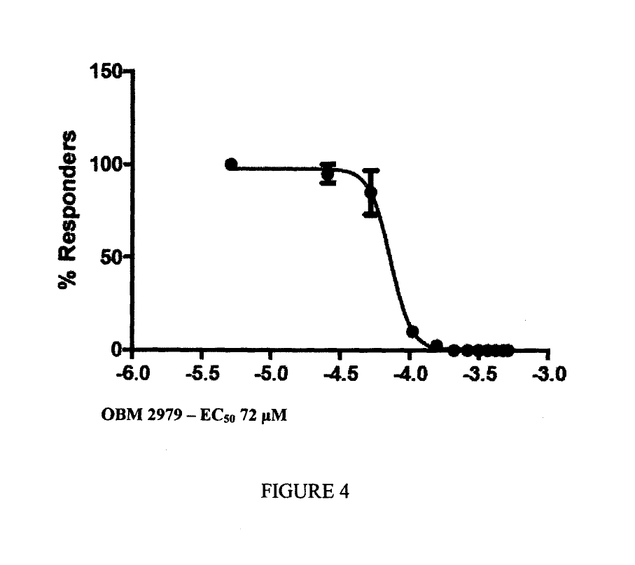

Briefly, the ZEA assay involved applying essential oils, fractions or individual compounds to developmentally staged zebrafish embryos followed by monitoring of embryonic touch response/swim behaviour and evaluation of the dose response relationship for each substance. Using a four point scale to describe the embryonic behaviours (Table 4), initial analysis focused on monitoring and recording these changes and evaluating the level of bioactivity. The effective concentrations to generate complete anaesthesia in 50% of the embryos (EC₅₀), were evaluated as follows:
Compounds were tested on developmentally staged AB “wild type” zebrafish embryos (54 hpf+/−2 hpf) at concentrations ranging between 10 and 400 μM.
Each compound was diluted in a 95% ethanol or DMSO carrier to create a working stock solution from which appropriate dilutions are made in standard embryo E3 media.
1000 μl of each concentration or appropriate carrier control was added to 10 wild type AB embryos in a single well of a 24 well plate, in duplicate.
The embryos were incubated for 90 min at 28° C. (optimal temperature for embryonic growth) in the diluted compound.
The four point scale (Table 4) was used to evaluate the touch response and swim behaviour for each embryo in all wells.
The effectiveness of the compound was based on its ability to generate complete anesthesia (scale: 1) in 50% of the embryos at a given concentration (EC₅₀).
The EC₅₀ values were calculated using GraphPad Prism® software to analyze the log (dose) response curves. The results are shown in Table 3, and in FIG. 3 as a dose response curve of zebrafish embryo assay, a percentage response versus percentage of compound present.

TABLE 3
Measured EC₅₀ values

| | | EC₅₀ |
|---|---|---|
| ID | Structure | (μM) |
| 2979 | 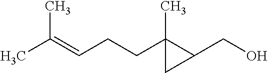 | B |
| 2989 | 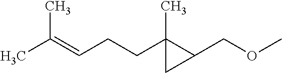 | D |
| 2993 | 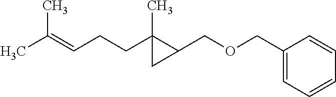 [SMILES: CC(C)=CCCC1(C)CC1COCc1ccccc1] | D |
| 2994 | 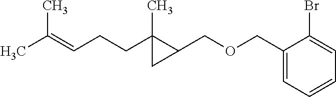 [SMILES: CC(C)=CCCC1(C)CC1COCc1ccccc1Br] | D |
| 2995 | 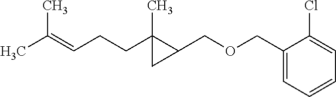 [SMILES: CC(C)=CCCC1(C)CC1COCc1ccccc1Cl] | D |
| 2999 | 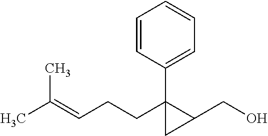 [SMILES: CC(C)=CCCC1(c2ccccc2)CC1CO] | C |

EC₅₀ ranges
A = 0.1-1 μM
B = 1-100 μM
C = 100-450 μM
D = >450 μM

TABLE 4
Four point scale representing 52-60hpf zebrafish embryonic behaviour

| Scale | Behaviour |
|---|---|
| 4 | Normal embryonic swim behaviour and touch response |
| 3 | Burst touch response with no swimming |
| 2 | Twitch response to touch |
| 1 | No observable touch response or swim behaviour |

### Example 6

### TRPV1 Assay Protocol—Calcium Imaging

Briefly, cells were seeded into poly-L-lysine-coated, glass-bottom, 24-well plates (1×10⁵ cells/well) and incubated overnight under standard culture conditions to achieve the desired confluency. Culture media was removed and cells were washed twice with HBS prior to incubation for 15 to 60 min at 37° C. with a labelling mixture comprised of Fura-2-AM and pluronic acid in HBS. Data collection occurred over an eight minute period and followed the same general sequence. Following loading, cells were stimulated by addition of 1 μM of capsaicin agonist for 2 min, after which a concentration series of the test sample (e.g. (0.5, 5, 10, 50 μg/ml) was added and imaging continued for an additional 5 min. Capsazepine (20 μM) served as a known reference antagonist, while cells that were mock-treated or received vehicle (e.g., DMSO) alone served as negative controls. For imaging, plates were placed on the stage of an inverted epifluorescence microscope (e.g., Axiovert 200, Zeiss) equipped with a CCD digital camera (e.g., Axiocam MRm, Zeiss). For each well of the plate, a sequence of image pairs (excitation at 340 nm and 380 nm) were collected to capture intracellular calcium flux. Image sequences were analyzed in ImageJ (NIH) and average pixel intensities calculated for six representative cells in each test condition to achieve mean fluorescence.

IC₅₀ results are shown in Table 5.

TABLE 5
IC₅₀ Results

| ID | Structure | IC₅₀ (μg/mL) |
|---|---|---|
| 2979 | 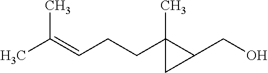 | agonist |
| 2989 | 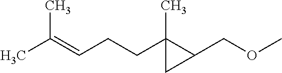 | — |
| 2993 | 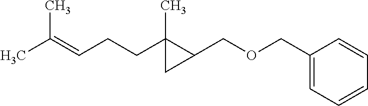 [SMILES: CC(C)=CCCC1(C)CC1COCc1ccccc1] | — |
| 2994 | 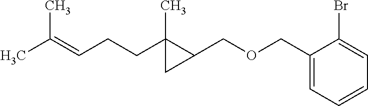 [SMILES: CC(C)=CCCC1(C)CC1COCc1ccccc1Br] | — |
| 2995 | 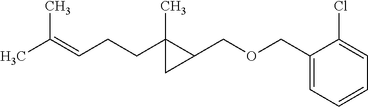 [SMILES: CC(C)=CCCC1(C)CC1COCc1ccccc1Cl] | — |
| 2999 | 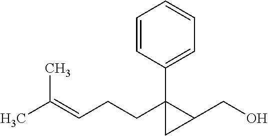 [SMILES: CC(C)=CCCC1(c2ccccc2)CC1CO] | — |

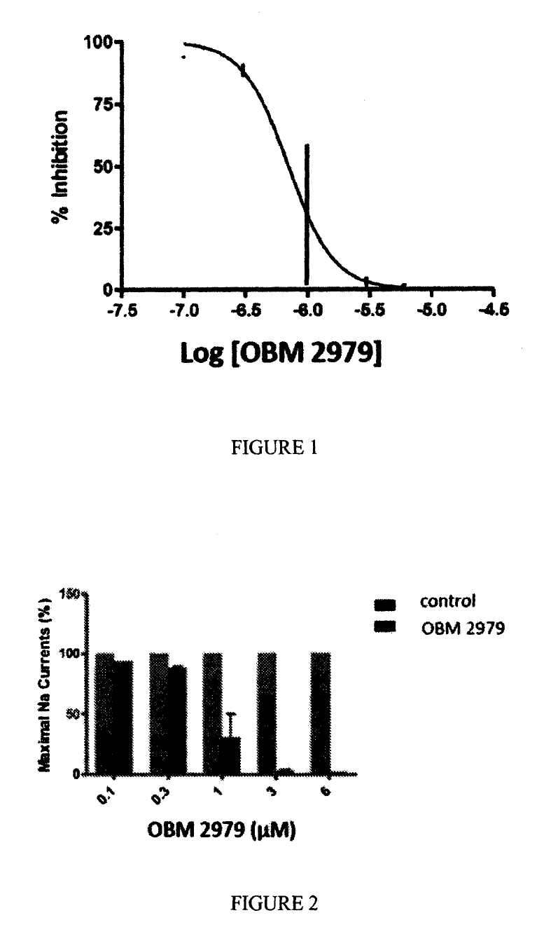

Results from testing using OBM 2979 at various concentrations in the presence of HEK-TRPV cells are shown in FIG. 2, with Ca²⁺ imaging.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A method of treating a neurological condition comprising administering to a human or animal a therapeutically effective amount of a terpene analogue of Formula 1: 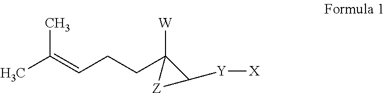
wherein:
Y is a substituted or unsubstituted C₁ to C₂₀ alkylene, C═O, SO, SO₂, or absent;
X is H, OR¹, N—(R²)₂, a substituted or unsubstituted C₁ to C₂₀ alkyl, or a substituted or unsubstituted heterocyclyl (for example, heteroaryl), wherein when Y is absent X is not H;
R¹ is H, a substituted or unsubstituted C₁ to C₂₀ alkyl, or a substituted or unsubstituted CH₂-aryl;
each R² is independently H, a substituted or unsubstituted C₁ to C₂₀ alkyl, aryl, OR¹, CN or C(═O)—R³;
R³ is a substituted or unsubstituted C₁ to C₂₀ alkyl, or a substituted or unsubstituted aryl;
W is H, a substituted or unsubstituted C₁ to C₂₀ alkyl, or a substituted or unsubstituted aryl; and
Z is a substituted or unsubstituted C₁ to C₂₀ alkylene;
or a pharmaceutically acceptable isomer, salt or ester thereof.

2. The method of claim 1, wherein Y is CH₂, W is CH₃, Z is CH₂ and X is OH, OCH₃, OCH₂-aryl.

3. The method of claim 1, wherein Y is CH₂, X is OH, W is phenyl, and Z is CH₂.

4. The method of claim 1, wherein the terpene analogue is a compound of Formula 1a: 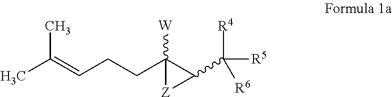
wherein:
R⁴ is OH, alkoxyl, aryloxyl, —NH₂, —SO₂Aryl, —SO₂NHAryl, —NHSO₂Aryl, —NHalkyl, —N(alkyl)₂, or —NHCO-Aryl;
W, R⁵, and R⁶ are each independently H, a substituted or unsubstituted C₁ to C₂₀ alkyl, a substituted or unsubstituted aryl or a substituted or unsubstituted alkylaryl; and
Z is a substituted or unsubstituted C₁ to C₂₀ alkylene.

5. The method of claim 1, wherein:
Y is a absent;
X is —C(═O)H, —COOH, —SO₂Aryl, or —SO₂NHAryl,
W is H, a substituted or unsubstituted C₁ to C₂₀ alkyl, a substituted or unsubstituted aryl or a substituted or unsubstituted alkylaryl; and
Z is a substituted or unsubstituted C₁ to C₂₀ alkylene.

6. The method of claim 1, wherein the terpene analogue is (2-methyl-2-(4-methylpent-3-en-1-yl)cyclopropyl)methanol, 2-(methoxymethyl)-1-methyl-1-(4-methylpent-3-en-1-yl)cyclopropane, (((2-methyl-2-(4-methylpent-3-en-1-yl)cyclopropyl)methoxy)methyl)benzene, 1-bromo-2-(((2-methyl-2-(4-methylpent-3-en-1-yl)cyclopropyl)methoxy)methyl)benzene, 1-chloro-2-(((2-methyl-2-(4-methylpent-3-en-1-yl)cyclopropyl)methoxy)methyl)benzene, or (2-(4-methylpent-3-en-1-yl)-2-phenylcyclopropyl)ethanol

7. The method of claim 1, wherein the terpene analogue is formulated for intravenous, topical, oral, intranasal, per rectal, intra muscular, intra dermal, intra vaginal, or subcutaneous administration.

8. The method of claim 1, wherein the neurological condition is pain.

9. The method of claim 8, wherein the pain is neuropathic pain.

10. A composition for treating a neurological condition, comprising a terpene analogue of Formula 1: 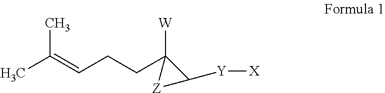
wherein:
Y is a substituted or unsubstituted C₁ to C₂₀ alkylene, C═O, SO, SO₂, or absent;
X is H, OR¹, N—(R²)₂, a substituted or unsubstituted C₁ to C₂₀ alkyl, or a substituted or unsubstituted heterocyclyl (for example, heteroaryl), wherein when Y is absent X is not H;
R¹ is H, a substituted or unsubstituted C₁ to C₂₀ alkyl, or a substituted or unsubstituted CH₂-aryl;
each R² is independently H, a substituted or unsubstituted C₁ to C₂₀ alkyl, aryl, OR¹, CN or C(═O)—R³;
R³ is a substituted or unsubstituted C₁ to C₂₀ alkyl, or a substituted or unsubstituted aryl;
W is H, a substituted or unsubstituted C₁ to C₂₀ alkyl, or a substituted or unsubstituted aryl; and
Z is a substituted or unsubstituted C₁ to C₂₀ alkylene;
or a pharmaceutically acceptable isomer, salt or ester thereof, and,
optionally, a pharmaceutically acceptable diluent or carrier.

11. The composition of claim 10, wherein Y is CH₂, W is CH₃, Z is CH₂, and X is OH, O—CH₃, or O—CH₂-aryl

12. The composition of claim 10, wherein Y is CH₂, X is OH, W is phenyl, and Z is CH₂.

13. The composition of claim 10, wherein the terpene compound is selected from the group consisting of (2-methyl-2-(4-methylpent-3-en-1-yl)cyclopropyl)methanol, 2-(methoxymethyl)-1-methyl-1-(4-methylpent-3-en-1-yl)cyclopropane, (((2-methyl-2-(4-methylpent-3-en-1-yl)cyclopropyl)methoxy)methyl)benzene, 1-bromo-2-(((2-methyl-2-(4-methylpent-3-en-1-yl)cyclopropyl)methoxy)methyl)benzene, 1-chloro-2-(((2-methyl-2-(4-methylpent-3-en-1-yl)cyclopropyl)methoxy)methyl)benzene, (2-(4-methylpent-3-en-1-yl)-2-phenylcyclopropyl)methanol, and combinations thereof.

14. The composition of claim 10, wherein the terpene analogue is a compound of Formula 1a: 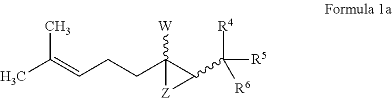
wherein:
R⁴ is OH, alkoxyl, aryloxyl, —C(═O)H, —COOH, —NH₂, —SO₂Aryl, —SO₂NHAryl, —NHSO₂Aryl, —NHalkyl, —N(alkyl)₂, or —NHCO-Aryl;
W, R⁵, and R⁶ are each independently H, alkyl, aryl or alkylaryl, where alkyl is C₁ to C₂₀; and
Z is a C₁ to C₂₀ alkylene.

15. The composition of claim 10, which is in a form for intravenous, topical, oral, intranasal, per rectal, intra muscular, intra dermal, intra vaginal, or subcutaneous administration.

16. The composition of claim 10, wherein the neurological condition is pain.

17. The composition of claim 16, wherein the pain is neuropathic pain.

18. Use of a terpene analogue of Formula 1: 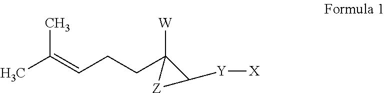
wherein:
Y is a substituted or unsubstituted C₁ to C₂₀ alkylene, C═O, SO, SO₂, or absent;
X is H, OR¹, N—(R²)₂, a substituted or unsubstituted C₁ to C₂₀ alkyl, or a substituted or unsubstituted heterocyclyl (for example, heteroaryl), wherein when Y is absent X is not H;
R¹ is H, a substituted or unsubstituted C₁ to C₂₀ alkyl, or a substituted or unsubstituted CH₂-aryl;
each R² is independently H, a substituted or unsubstituted C₁ to C₂₀ alkyl, aryl, OR¹, CN or C(═O)—R³;
R³ is a substituted or unsubstituted C₁ to C₂₀ alkyl, or a substituted or unsubstituted aryl;
W is H, a substituted or unsubstituted C₁ to C₂₀ alkyl, or a substituted or unsubstituted aryl; and
Z is a substituted or unsubstituted C₁ to C₂₀ alkylene;
or a pharmaceutically acceptable isomer, salt or ester thereof,
for treating a neurological condition in a subject in need thereof.

19. The use according to claim 18, wherein Y is CH₂, W is CH₃, Z is CH₂ and X is OH, OCH₃, OCH₂-aryl

20. The use according to claim 18, wherein Y is CH₂, X is OH, W is phenyl, and Z is CH₂.

21.-29. (canceled)
